(19) United States
(12) Patent Application Publication
(10) Pub. No.: US 2011/0190706 A1
(43) Pub. Date: Aug. 4, 2011

(54) Angio Catheter with Improved Safety Features

(76) Inventors: William Paul Spurbeck, Tucson, AZ (US); Mark Banister, Tucson, AZ (US)

(21) Appl. No.: 13/014,992

(22) Filed: Jan. 27, 2011

Related U.S. Application Data
(60) Provisional application No. 61/337,155, filed on Feb. 1, 2010.
(60) Provisional application No. 61/406,016, filed on Oct. 22, 2010.

(51) Int. Cl. A61M 25/06; A61M 25/00

(52) U.S. Cl. 604/164.08; 604/537; 604/167.03

(57) ABSTRACT

An angiocatheter device featuring a catheter body; a stationary anchor disposed in the inner cavity of the catheter body; a channel fluidly connecting the first end of the catheter body and the second end of the stationary anchor; and a compressible valve surrounding at least a portion of the stationary anchor. A hole is disposed in the second end of the compressible valve. The compressible valve can move between an extended and compressed position. In the extended position the second end of the compressible valve covers the second end of the stationary anchor and the hole in the compressible valve is closed preventing fluid transfer from the channel into the catheter body. In the compressed position the compressible valve is compressed such that the second ends of the stationary anchor and channel are accessible allowing for fluid transfer. The compressible valve is biased in the extended position.

### CROSS REFERENCE

This application claims priority to U.S. provisional application Ser. No. 61/337,155 filed Feb. 1, 2010 and U.S. provisional application Ser. No. 61/406,016 filed Oct. 22, 2010, the specification of which is incorporated herein by reference in its entirety.

### FIELD OF THE INVENTION

The present invention is directed to an angiocatheter device, more particular to an angiocatheter that utilizes a fluid retention component configured to stop any blood spill or leakage as the catheter is installed on the patient and connected to an intravenous (IV) line or other device. The present invention can also utilize needle safety mechanisms and enables freedom of the care providers hand normally occupied with stopping the blood flow out of the catheter.

### BACKGROUND OF THE INVENTION

Angiocatheters are devices that are inserted into a patient's vein for intravenous (IV) access for fluids and medications and in some cases obtain blood samples. Usually an angiocatheter is inserted by using the needle, which is positioned within the lumen of the cannula. When the needle penetrates the vessel, the blood pressure in the vessel will cause blood to flow up the needle bore and into translucent tubing around the top of the angiocatheter. The practitioner verifies the penetration of the vessel by looking for blood “flash” in the chamber. The cannula of the angiocatheter is then advanced within the blood vessel to a desired position and the needle is withdrawn from the cannula.

Sometimes when the needle is withdrawn and the practitioner attempts to introduce either IV tubing or a blood sampling device, blood may flow from the catheter, which may cause a pathogen exposure. This process for the practitioner can be difficult, since generally the practitioner must use one hand (once the catheter is installed) to press on the vein ahead of the inserted catheter to stop blood flow out of the catheter, thereby allowing the practitioner only one hand to unwrap and install a saline lock or IV line into the open end of the catheter from which blood can escape. This poses a risk and increases the chance that the practitioner is exposed to the patient's blood.

Current models of catheters and the catheters described herein may be manufactured in many ways but are typically injection molded out of polyethylene or other biologically safe thermoplastics well known in the art. The catheters may also be made of semi transparent, transparent or clear materials either in whole or in a portion of the catheter to allow the observation of the blood flash through the body of the catheter. Typically the soft or flexible cannula has a material embedded in it to make it visible via radiograph during or after insertion into the patient. These materials and methods to manufacture and embed in the cannula are well known in the art.

The present invention features a novel angiocatheter device that helps protect the practitioner, patient and equipment from unnecessary exposure and contamination. For example, the angiocatheter device of the present invention helps to stop the chance of a blood splash or leak that could possibly risk and expose the healthcare provider or patient to pathogens and diseases such as hepatitis or human immunodeficiency virus (HIV). Such a device can help provide a much safer work environment for healthcare providers and technicians.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY

The present invention features novel angiocatheter devices for helping to protect a practitioner, a patient, and equipment from exposure and contamination. In some embodiments, the device comprises a catheter body having a first end, a second end, and an inner cavity, the second end being open providing access to the inner cavity of the catheter body; a stationary anchor disposed in the inner cavity of the catheter body, a first end of the stationary anchor facing the first end of the catheter body and a second end of the stationary anchor facing the second end of the catheter body; a channel having a first end and a second end, the first end being positioned at the first end of the catheter body and the second end being positioned at the second end of the stationary anchor, the channel fluidly connects the first end of the catheter body and the second end of the stationary anchor; and a compressible valve, a first end of the compressible valve surrounds at least a portion of the stationary anchor and a second end faces the second end of the catheter body, a hole is disposed in the second end of the compressible valve.

The compressible valve can move between at least an extended position and a compressed position, wherein in the extended position the second end of the compressible valve is positioned closer to the second end of the catheter body than is the second end of the stationary anchor and the hole in the second end of the compressible valve is closed preventing fluid transfer from the channel into the catheter body, wherein in the compressed position the compressible valve is compressed such that the second end of the stationary anchor is closer to the second end of the catheter body than is the second end of the compressible valve and the hole is the second end of the compressible valve is open allowing fluid access to the second end of the channel and second end of the stationary anchor for fluid transfer, the compressible valve is biased in the extended position.

In some embodiments, the angiocatheter device further comprises a cannula disposed on the first end of the catheter body and fluidly connected to the channel. In some embodiments, the angiocatheter device further comprises a removable needle housed within the cannula. In some embodiments, the angiocatheter device further comprises a needle stick safety device engaged with the needle, the needle stick safety device is removed from device if the needle is removed.

In some embodiments, the angiocatheter device further comprises a lip disposed around the second end of the catheter body. In some embodiments, the angiocatheter device further comprises stabilizing tabs disposed on and protruding from sides of the catheter body.

In some embodiments, the catheter body is generally cone-shaped with the first end having a diameter smaller than that of the second end. In some embodiments, the catheter body is generally cylindrical in shape. In some embodiments, the angiocatheter device further comprises a window disposed in the catheter body. In some embodiments, the window is constructed from a translucent, transparent, or clear material. In some embodiments, the stationary anchor is generally cone-shaped with the second end having a diameter smaller than that of the first end. In some embodiments, the compressible valve is constructed from a material comprising a silicone, a rubber, a foam, or any flexible, compressible medical grade material that can be utilized as the compressible valve.

In some embodiments, the first end of the compressible valve is mounted around the stationary anchor via a mounting component. In some embodiments the mounting component for the compressible valve is a second, third or fourth component that slides into the catheter body after the catheter cannula and stationary anchor are assembled into the catheter. In some embodiments, the angiocatheter device further comprises a cover adapted to temporarily cover the second end of the hub. In some embodiments, the cover is attached to the catheter body via an attachment means. In some embodiments, the angiocatheter device further comprises a saline lock attached to the catheter body via an attachment means.

In some embodiments, the angiocatheter device comprises a catheter body having a first end, a second end, and a hub disposed on the second end of the catheter body, the connection hub has an open second end; a valve disposed in the hub of the catheter body; and a channel, the channel extending from the valve to at least the first end of the catheter body. The valve can move between an open position and a closed position and is biased in the closed position, wherein when a needle or a medical fitting is introduced into the hub of the catheter body via the open second end of the hub and makes contact with the valve the valve moves to the open position allowing fluid to transfer between the channel and the needle or medical fitting, and when the needle or medical fitting is removed from the valve the valve moves to the closed position preventing flow of fluid from channel into the catheter body.

In some embodiments, the valve is a reed-type valve, a tines valve, a clapper-type valve, a ball-type valve, a self-sealing membrane valve, a septum valve, a compressible valve, or a combination thereof. In some embodiments, the septum valve is constructed from but not limited to a material comprising a silicone, a rubber, a foam, or combinations of these. The septum may be made of any compressible material that is biocompatible and can be sterilized as well as approved for medical use.

In some embodiments, the angiocatheter device further comprises a cannula disposed on the first end of the catheter body and fluidly connected to the channel. In some embodiments, the angiocatheter device further comprises a removable needle housed within the cannula. In some embodiments, the angiocatheter device further comprises a needle stick safety device engaged with the needle, the needle stick safety device is removed from device if the needle is removed.

In some embodiments, the angiocatheter device further comprises stabilizing tabs disposed on and protruding from sides of the catheter body. In some embodiments, the angiocatheter device further comprises a window disposed in the catheter body. In some embodiments, the angiocatheter device further comprises a cover adapted to temporarily cover the second end of the hub. In some embodiments, the cover is attached to the catheter body via an attachment means. In some embodiments, the angiocatheter device further comprises a saline lock attached to the catheter body via an attachment means.

### BRIEF DESCRIPTION OF THE DRAWINGS

FIG. 1 is a side cross sectional view of an angiocatheter of the present invention. The compressible valve is in the extended position.

FIG. 2 is a side cross sectional view of an angiocatheter of the present invention, wherein a syringe is partially inserted into the catheter body and the compressible valve is partially compressed.

FIG. 3 is a side cross sectional view of an angiocatheter of the present invention, wherein a syringe is partially inserted into the catheter body and the compressible valve is partially compressed.

FIG. 4 is a side cross sectional view of an angiocatheter of the present invention, wherein a syringe is inserted into the catheter body and the compressible valve is in the compressed position.

FIG. 5 is a side view of an alternative embodiment of the angiocatheter of the present invention.

FIG. 6 is a side view of an alternative embodiment of the angiocatheter of the present invention comprising a reed-type valve.

FIG. 7A is a side view of an alternative embodiment of the angiocatheter of the present invention comprising a tines valve.

FIG. 7B is an end view of the tines valve of FIG. 7A in the closed position.

FIG. 8A is a side view of an alternative embodiment of the angiocatheter of the present invention comprising a clapper-type valve.

FIG. 8B is a side view of the angiocatheter of FIG. 8A before the needle and needle stick prevention device are removed.

FIG. 9A is a side view of an alternative embodiment of the angiocatheter of the present invention, wherein an end cap is attached.

FIG. 9B is a side view of the angiocatheter of FIG. 9A wherein a saline lock is inserted into the connection hub.

FIG. 10A is a side view of an alternative embodiment of the angiocatheter of the present invention comprising a septum-type valve that closes when the needle is pulled out and can re-open when a standard male luer lock is attached to the catheter by pushing the septum valve over the stationary anchor.

FIG. 10B is a side and end views of the septum valve of the angiocatheter of FIG. 10A.

### DESCRIPTION OF PREFERRED EMBODIMENTS

In the following detailed description of the preferred embodiments, reference is made to the accompanying drawings, which form a part hereof, and in which is shown by way of illustration specific embodiments in which the invention may be practiced. In this regard, directional terminology, such as “upper”, “lower”, “side”, “top,” “bottom,” “front,” “back,” “leading,” “trailing,” etc., is used with reference to the orientation of the Figures being described. Because components of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense.

Details in the various embodiments such as how the catheters are molded of plastic and joined together via glue or RF welding or assembled with lumen cannula over the needle and how the needle is inserted into the plastic injection molded body of the catheter are all well known in the art.

The term “valve” refers to and includes all types of valves that could be incorporated into the catheter housing to stop the flow of blood back out of the catheters exposed end. Samples of these are spring-loaded clapper valves, spring-loaded ball valves, constant tension activated valves, reed valves, pierceable membranes, self-sealing membranes, compressible septum valves such as those used to seal vials. These are non-limiting examples of various valves or liquid containment mechanisms that would allow repeated access to a patient's blood without allowing the blood to spill or flow back out of the catheter unless desired. These various examples including those that are generally well known in the art are not meant to limit the scope of the invention in any way.

The term needle stick safety device, refers to any component, mechanism or attachment to the catheter designed to prevent accidental needle sticks once the needle is pulled out of or removed from the catheter, there are many examples of these that are well known in the art.

The cannula refers to the sheath or tubing portion of the catheter that covers the needle and is left in the vein once the needle is removed. The cannula is attached to the housing or body of the catheter even when the needle is removed or pulled out of the catheter. The cannula may be constructed from a material comprising a biocompatible flexible plastic or polymer tubing that is well known in the art, however the cannula is not limited to these materials.

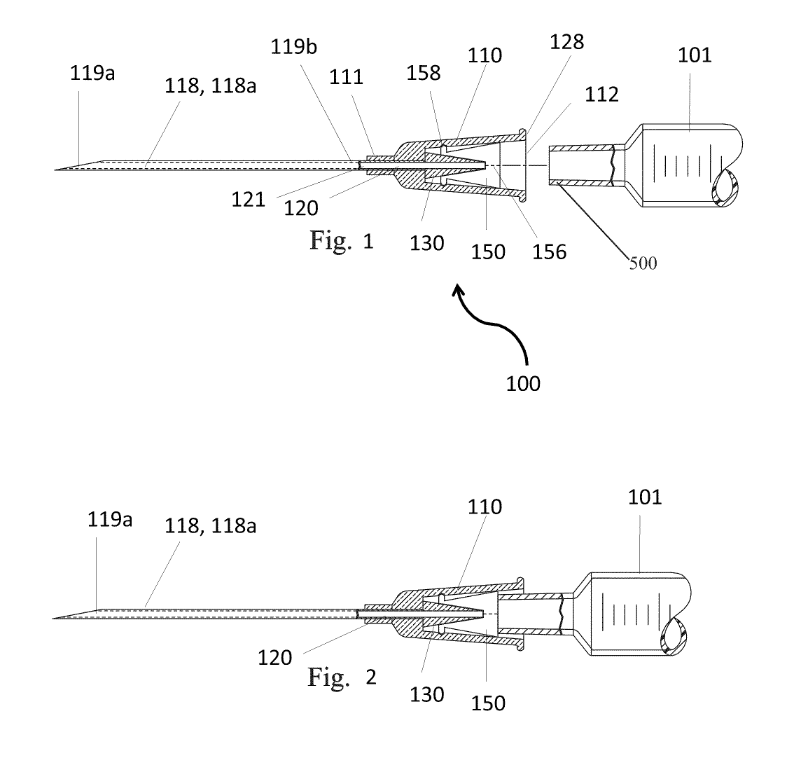

Referring now to FIG. 1-10, the present invention features a novel angiocatheter device that helps to stop the chance of a blood splash or leak that could possibly expose a healthcare provider or patient to pathogens.

As shown in FIG. 1-4, the angiocatheter device 100 comprises a catheter body 110 (e.g., a generally hollow catheter body 110) having a first end 111 and a second end 112. The first end 111 of the catheter body 110 is connected (e.g., fluidly connected) to the cannula 118 (with the needle 118a optionally inserted through the cannula 118) and the second end 112 engages a syringe 101, IV system, blood collection tube, or the like. Generally, the first end 119a of the cannula 118 (or needle 118a) is the portion that is inserted and anchored into the patient's vessel. The second end 119b of the cannula 118 attaches to the first end 111 of the catheter body 110 (in some embodiments, the second end 119b of the cannula 118 extends into the inner cavity of the catheter body 110 via the first end 111 of the catheter body 110). The second end 112 of the catheter body 110 is generally open, providing access to the inner cavity of the catheter body 110. The catheter body 110 may be generally cone shaped or cylindrical in shape, however the catheter body 110 is not limited to the aforementioned shapes. In some embodiments, a lip 128 is disposed around the second end 112 of the catheter body 110. The lip 128 may provide a surface with which a syringe 101, IV system, or blood collection tube can engage to secure the catheter body 110.

In some embodiments, a stationary anchor 130 is disposed in the inner cavity of the catheter body 110. The stationary anchor 130 has a first end and a second end 132. In some embodiments, the stationary anchor 130 is generally cone shaped with the second end 132 being the end with the smaller diameter. The stationary anchor 130 is not limited to being cone shaped, for example the stationary anchor 130 may be generally cylindrical in shape or irregular in shape, or the like. The first end of the stationary anchor 130 may be mounted at or near the first end 111 of the catheter body 110 (e.g., see FIG. 1) and the second end 132 of the stationary anchor 130 faces towards the second end 112 of the catheter body 110.

In some embodiments, a channel 120 is disposed in the stationary anchor 130, wherein the first end 121 of the channel 120 is positioned at the first end 111 of the cannula body 110 and the second end 122 of the channel 120 is positioned at the second end 132 of the stationary anchor 130 (see FIG. 1). The channel 120 fluidly connects the first end 111 of the catheter body 110 to the second end 132 of the stationary anchor 130. In some embodiments, a cannula 118 (and/or needle 118a) is attached (or is insertable, removably attached, etc.) to the first end 111 of the catheter body and is fluidly connected to the channel 120. In some embodiments, a portion of the cannula 118 (e.g., the second end 119b) traverses the stationary anchor 130, wherein the second end 119b of the cannula 118 is positioned at the second end 132 of the stationary anchor 130. The cannula 118 allows fluid to pass to or from the second end 132 of the stationary anchor 130 through the cannula 118 (and/or needle 118a). For example, the cannula 118 fluidly connects the second end 132 of the stationary anchor 130 to the first end 111 of the catheter body 110 (and typically the first end 119a of the cannula 118). The cannula 118 typically extends beyond the first end 111 of the catheter body 110, however the device 100 of the present invention may not necessarily be constructed with a full cannula 118. Or, in some embodiments, a cannula or cannula extension can be attached to the first end 111 of the catheter body 110.

Mounted to the stationary anchor 130 is a compressible valve 150. The compressible valve 150 has a first end and a second end 152. In some embodiments, the compressible valve 150 is generally cone shaped, wherein the first end is the end with the smaller diameter. The compressible valve 150 is not limited to being cone shaped, for example the compressible valve 150 may be generally cylindrical in shape or irregular in shape, or the like. The first end of the compressible valve 150 may be mounted around the stationary anchor 130 (e.g., via a mounting component 158) at or near the first end or middle portion of the stationary anchor 130. In some embodiments, an indentation is disposed in the first end of the compressible valve 150 adapted to accept the second end 132 of the stationary anchor 130 (e.g., the compressible valve 150 fits over the second end 132 of the stationary anchor 130). A slit or tiny hole 156 is disposed in the second end 152 of the compressible valve 150.

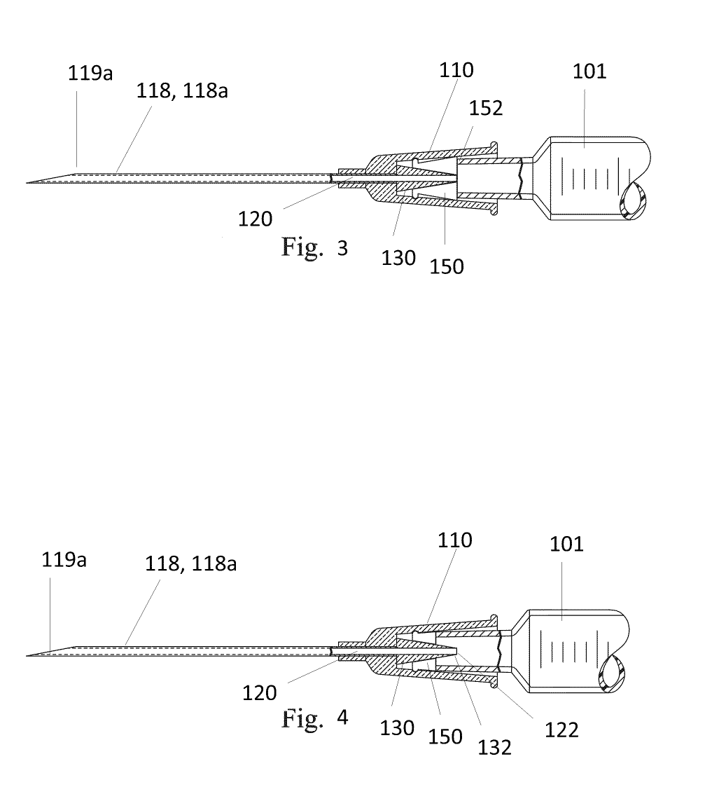

The compressible valve 150 can be compressed, thus the compressible valve 150 can move between multiple positions including an extended position and a compressed position, wherein the compressible valve 150 is biased in the extended position caused by the material of the compressible valve 150. In the extended position, as shown in FIG. 1, the second end 152 of the compressible valve 150 is positioned closer to the second end 112 of the catheter body 110 than is the second end 132 of the stationary anchor 130. The hole 156 of the compressible valve 150 is closed (e.g., by way of the nature of the material of the compressible valve 150). In the extended position, the hole 156 is closed and the compressible valve 150 blocks access to the channel 120, e.g., the second end 122 of the channel 120, (and optionally the needle 118a and/or cannula 118), thereby preventing blood from the vessel from leaking into the catheter body 110. In the compressed position, as shown in FIG. 4, the compressible valve 150 is compressed such that the second end 132 of the stationary anchor 130 is closer to the second end 112 of the catheter body 110 than is the second end 152 of the compressible valve 150. The second end 132 of the stationary anchor 130 is effectively exposed through the hole 156 of the compressible valve 150 (which is opened and compressed when the compressible valve 150 is in the compressed position). In the compressed position a syringe, IV system, or blood collection tube can access the channel 120 (e.g., and optionally the needle 118a and/or cannula 118) via the second end 132 of the stationary anchor 130 for fluid transfer. FIG. 4 shows a syringe 101 fluidly connected to the channel 120. FIG. 2 and FIG. 3 show positions in between the extended position and the compressed position. The compressible valve 150 may be compressed by a compressing component 500, for example a syringe tip. In some embodiments, the compressible valve 150 is biased in the extended position and will return to the biased extended position from the compressed position after a compressing component 500 is no longer pressing onto the compressive valve 150.

The compressible valve 150 is constructed from a material comprising a compressible material, for example a silicone, a rubber, a thick or dense foam, a foam-like rubber material, or the like. Other materials may include polymers such as polyethylene blends, silicone co-polymers, block polymers or other elastic compressible materials that can be sterilized and are biocompatible or approved for medical use.

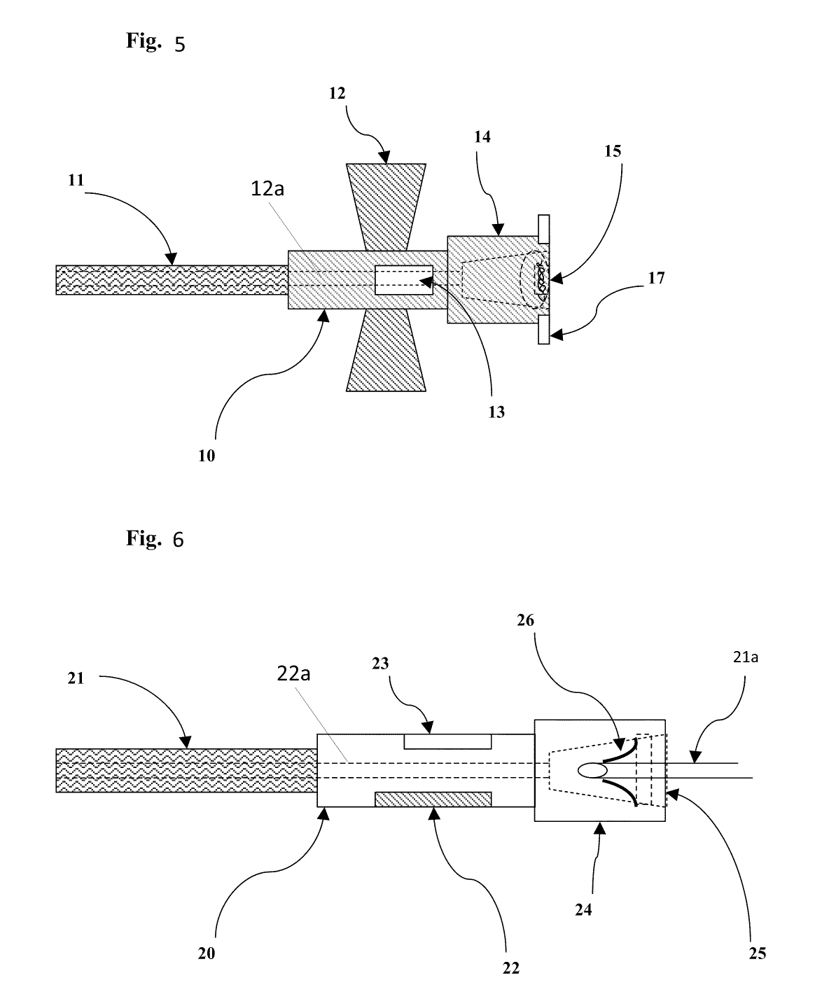

In some embodiments, a window (e.g., a blood flash window) (shown in FIG. 5, not shown in FIG. 1-4) is disposed in the catheter body 110, which allows for visualization of blood flash to confirm that the practitioner has fluidly connected the device to the vein appropriately. For example, a portion of the catheter body 110 may be constructed from a translucent, transparent, or clear material. Optionally, other portions of the catheter body 110 (or all of the catheter body 110) may be constructed from a translucent, transparent, or clear material. As used herein, the terms “clear,” “translucent,” and “transparent” refer to a property of a material that allows visualization of light, an object, or a shadow.

In some embodiments, stabilizing tabs (shown in FIG. 5, not shown in FIG. 1-4) are disposed on and protrude from the sides of the catheter body 110. The stabilizing tabs can help prevent turning or twisting of the device 100 once the device 100 is inserted and secured in the patient.

As shown in FIG. 5, the angiocatheter device of the present invention comprises a catheter body 10 (having a first end and a second end). The catheter body 10 allows fluid connectivity throughout the device (except when a valve is closed to stop blood flow). Disposed in the catheter body 10 is a blood flash window 13, which allows for visualization of blood flash to confirm that the practitioner has fluidly connected the device to the vein appropriately. The window 13 may be constructed from a clear, translucent, or transparent material. Optionally, the remaining portions of the catheter body 10 may be constructed from a less transparent, clear, or translucent material in order to make visual confirmation that the blood vessel has been penetrated as easy as possible for the health care provider.

A channel 12a is disposed in the catheter body 10, which can be fluidly connected to the cannula 11. The cannula 11 (e.g., the component inserted/anchored into the patient's vessel, may be constructed from a biologically compatible plastic material. The cannula 11 may be attached to the first end of the catheter body (e.g., via a welding, molding or gluing process, etc.) and be fluidly connected to the channel 12a (the channel 12a extends into the inner cavity of the catheter body 10/hub 14).

The device further comprises a connection hub 14 (having a first end and a second end 15), which may be a molded part of the catheter body 10 (e.g., FIG. 1-4 show the connection hub 14 as being part of the catheter body 10). For example, the hub 14 is a part of the catheter body 10 at the second end of the catheter body 10. The connection hub 14 is generally hollow with an open second end 15. The connection hub 14 allows for connection of the medical (e.g., IV) tubing or syringe (or other fitting such as a luer lock system). In some embodiments, connection hub tabs 17 are disposed on the outer edge of the second end of the connection hub 14 (e.g., similar to the lip 128 on the second end 112 of the catheter body 110 in FIG. 1-4). The connection hub tabs 17 may engage the medical tubing or syringe or other fitting.

In some embodiments, stabilizing tabs 12 are disposed on and protrude from the sides of the catheter body 10. The stabilizing tabs 12 can help prevent turning or twisting of the device once the device is inserted and secured in the patient.

The embodiment shown in FIG. 6 includes a reed-type valve 26 (e.g., constructed from a biocompatible plastic, optionally with fiber reinforcement). The needle 21a can be pulled out of the catheter body 20 and hub 24 once the device (e.g., the cannula) is inserted and anchored in a patient's vessel (e.g., the flexible cannula 21 has penetrated the vessel). When the needle 21a is pulled out, the reed-type valve 26 closes tightly behind it (preventing flow of blood from the vessel into the catheter body 20 and hub 24). A needle can be reinserted (through the reed-type valve 26) to access fluid or blood for samples or to administer a medication or other fluid, for example the needle 21a can penetrate the opening at the second end 25 of the hub 24. Additionally shown in FIG. 6 are the flash window 23 and the stabilizing wings 22. A channel 22a is disposed in the catheter body 20 and is fluidly connected to the cannula 21.

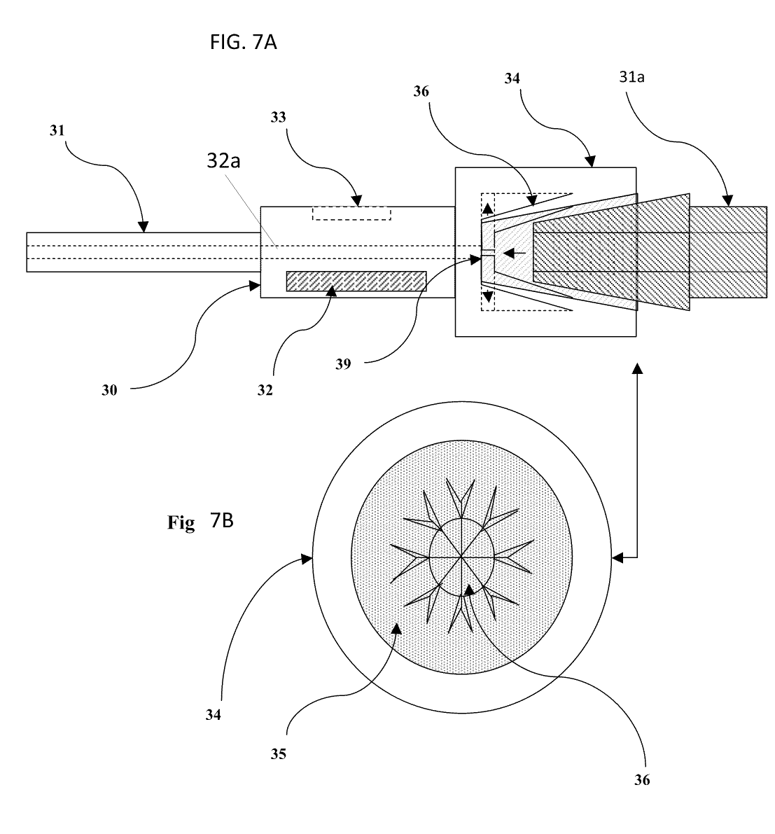

The embodiments shown in FIGS. 7A and 7B include a pinching finger-like tines valve 36. The tines valve 36 may be constructed from a material comprising a biocompatible plastic, however the valve 36 is not limited to the aforementioned materials. The needle (previously inserted into the device) holds the tines or fingers of the tines valve 36 open until the needle can be pulled out of the catheter body 30 and hub 34. Once the device is inserted/anchored in a patient's vessel (e.g., with the needle and cannula 31), the needle is pulled out and the tines valve 36 closes tightly behind it. A needle may not necessarily be able to be reinserted to access fluid or blood for samples. This may be accomplished by using a standard medical fitting 31a, which when pushed in to the open second end 35 of the hub 34 pushes the fingers or tines of the tines valve 36 to the open position 39 allowing blood vessel access and fluid connection through the catheter. The tines and hub may be manufactured for example, but not limited to, by injection molding as two parts to be welded or glued together later in the assembly process. Additional components may include stabilizing tabs 32 and a blood flash window 33. The window 33 is shown on the catheter body 30 in FIG. 7A, however the window 33 may alternatively located on the hub 34. A channel 32a is disposed in the catheter body 30 and is fluidly connected to the cannula 31.

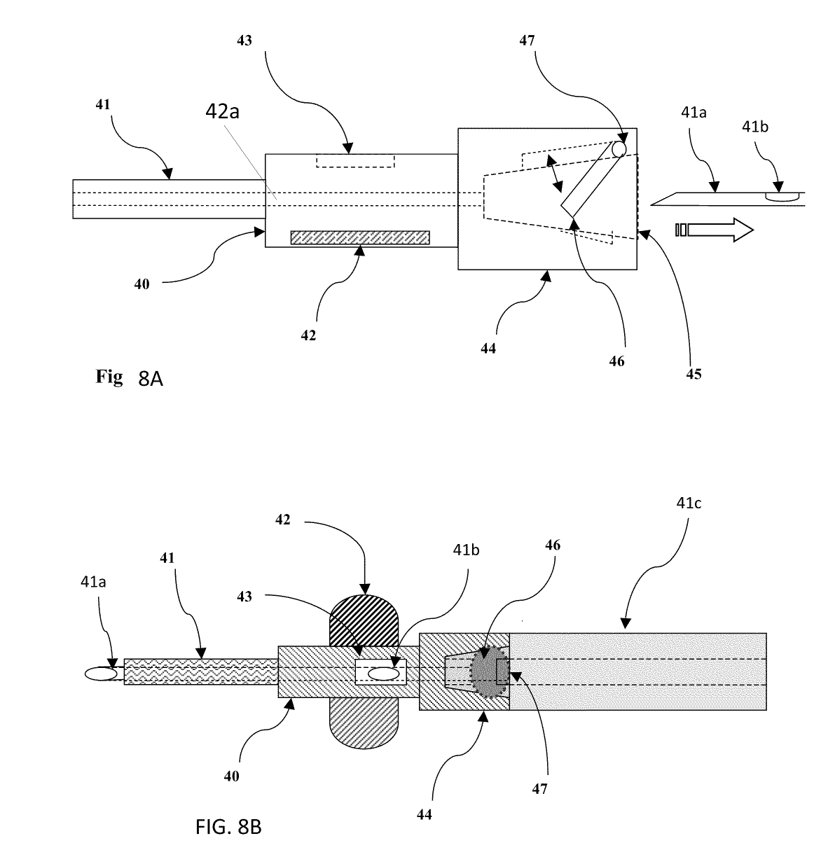

The embodiment shown in FIG. 8A shows the device with a spring-tensioned clapper-type valve device 46 that closes and seals the open second end 45 of the hub 44 as the needle 41a is pulled out of the cannula 41, catheter body 40, and catheter hub 44. The spring 47 of the spring-tensioned clapper-type valve 46 allows the valve 46 to move to the closed position when the needle 41a is removed (e.g. the spring 47 biases the valve 46 in the closed position). Also shown in FIG. 8A is the flash window 43 in the catheter body 40 and the needle 41a with an opening 41b in the needle 41a. Also shown in FIG. 8A are the stabilizing tabs 42. A channel 42a is disposed in the catheter body 40 and is fluidly connected to the cannula 41.

FIG. 8B shows the embodiment of FIG. 8A with a fully inserted needle 41a and a needle stick safety device 41c that retracts the needle 41a into its body to protect care providers from accidental needle sticks. When fully assembled the needle stick safety device 41c holds the spring-tensioned clapper-type valve 46 open in a recessed position within the catheter hub 44. The needle 41a is fully extended through the catheter body 40 and cannula 41 with the needle opening 41b aligned with the flash window 43. The tip of the needle 41a is exposed to enable penetration of the blood vessel.

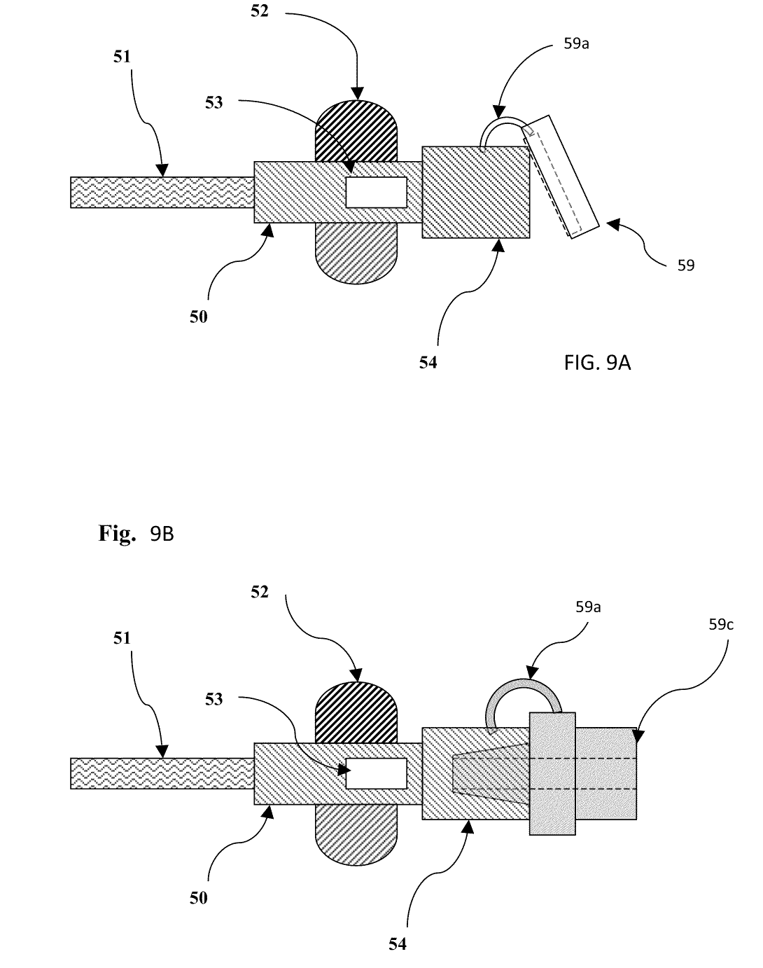

The embodiment shown in FIG. 9A shows the catheter body 50, cannula 51, stabilizing tabs 52, flash window 53, hub 54, and a cover 59 (e.g., removable cover 59) that can be snapped or pushed over the open second end of the hub 54 to help keep it sterile. The cover 59 may be attached to the hub 54 by an attachment means 59a (e.g., a flexible plastic strap, tether, link, or the like) so that it is readily available for the care provider. Alternatively FIG. 9B shows a saline lock 59c attached to the hub 54 via an attachment means 59a (e.g., a flexible plastic strap, tether, link, or the like), again to enable easy access to the saline lock 59c and also allow a care provider to use one hand to install the saline lock 59c into the device. Both embodiments in FIG. 9A and FIG. 9B may be used with or without a valve (e.g., reed-type valve 26, spring-tensioned clapper-type valve 46, tines valve 36, compressible valve 150, etc.) in the catheter.

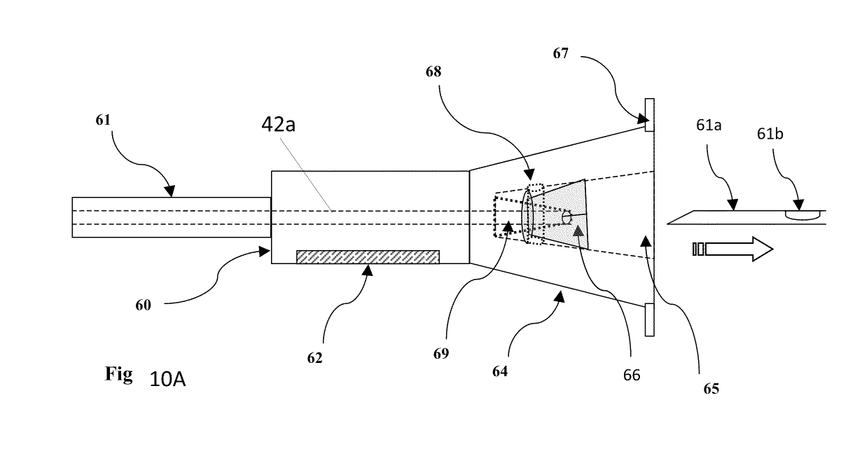

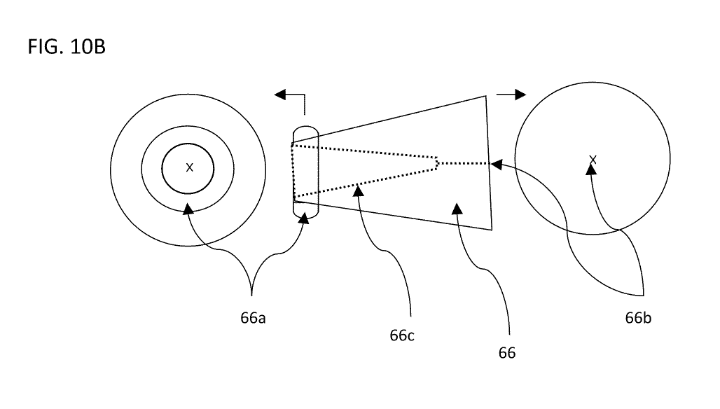

The embodiment shown in FIG. 10A is similar to the embodiments shown in FIG. 1-4. For example, FIG. 10A shows the catheter body 60 (e.g., a semitransparent catheter body 60), the cannula 61, the stabilizing tabs 62, the hub 64 with an open second end 65, and a restricted septum-type valve 66 disposed in the hub 64. A channel 42a is disposed in the catheter body 60 and is fluidly connected to the cannula 61. In some embodiments, a stationary cone 69 is disposed in the hub 64, and the septum valve 66 is attached to the stationary cone 69 via a mounting component 68 (e.g., a molded ridge). The septum valve 66 closes when the needle 61a (e.g., optionally with a flash opening 61b) is removed (e.g., FIG. 10A shows the valve 66 in the closed position wherein access cannot be gained to the cannula 61 because the valve 66 is covering the cannula 61 opening). The septum valve 66 can be opened (moved to the open position) when it is compressed, for example when a male fitting is inserted or locked into the hub (optionally utilizing locking tabs 67 on the second end 65 of the hub 64). The male end is pressed against the septum valve, which in turn forces the septum valve to compress and ride over the stationary cone 69.

FIG. 10B shows a side and detailed view of the septum valve 66 of FIG. 10A. A ridge 66a helps retain the septum valve 66 when in the device. Also shown is a recessed area or cavity 66c disposed in the septum valve 66 adapted to accept the stationary anchor 69 (also stationary anchor 130) in the hub 64. A slit or small hole 66b is disposed ion the septum valve 66. The slit or hole 66b allows the fluidic connectivity when opened (e.g., when the valve 66 is compressed, fluid access to the cannula can be obtained via the hole 66b.

The septum valve may be constructed from a material comprising a biologically safe elastomeric material or combinations of materials such as, but not restricted to, silicone, rubber or polyurethane so that the material returns to its original shape after it is compressed over the stationary anchor 69 and then released or allowed to relax (e.g., once a syringe, luer lock type device, or other medical connection is removed thus allowing needle-less access or fluid connectivity to the catheter and patient using standard medical devices, such as but not limited to, a needle-less syringe or IV tubing connections).

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the an that modifications may be made thereto which do not exceed the scope of the appended claims. Therefore, the scope of the invention is only to be limited by the following claims.

The reference numbers recited in the below claims are solely for ease of examination of this patent application, and are exemplary, and are not intended in any way to limit the scope of the claims to the particular features having the corresponding reference numbers in the drawings.

What is claimed is:

1. An angiocatheter device 100 comprising:
(a) a catheter body 110 having a first end 111, a second end 112, and an inner cavity, the second end 112 being open providing access to the inner cavity of the catheter body 110;
(b) a stationary anchor 130 disposed in the inner cavity of the catheter body 110, a first end of the stationary anchor 130 facing the first end 111 of the catheter body 110 and a second end 132 of the stationary anchor 130 facing the second end 112 of the catheter body 110;
(c) a channel 120 having a first end 121 and a second end 122, the first end 121 being positioned at the first end 111 of the catheter body 110 and the second end 122 being positioned at the second end 132 of the stationary anchor 130, the channel 120 fluidly connects the first end 111 of the catheter body 110 and the second end 132 of the stationary anchor 130; and
(d) a compressible valve 150, a first end of the compressible valve 150 surrounds at least a portion of the stationary anchor 130 and a second end 152 faces the second end 112 of the catheter body 110, a hole 156 is disposed in the second end 152 of the compressible valve 150;
wherein the compressible valve 150 can move between at least an extended position and a compressed position, wherein in the extended position the second end 152 of the compressible valve 150 is positioned closer to the second end 112 of the catheter body 110 than is the second end 132 of the stationary anchor 130 and the hole 156 in the second end 152 of the compressible valve 150 is closed preventing fluid transfer from the channel 120 into the catheter body 110, wherein in the compressed position the compressible valve 150 is compressed such that the second end 132 of the stationary anchor 130 is closer to the second end 112 of the catheter body 110 than is the second end 152 of the compressible valve 150 and the hole 156 is the second end 152 of the compressible valve 150 is open allowing fluid access to the second end 122 of the channel 120 and second end 132 of the stationary anchor 130 for fluid transfer, the compressible valve 150 is biased in the extended position and will return to the biased extended position from the compressed position after a compressing component 500 is no longer pressing onto the compressive valve 150.

2. The angiocatheter device 100 of claim 1 further comprising a cannula 118 disposed on the first end 111 of the catheter body and fluidly connected to the channel 120.

3. The angiocatheter device 100 of claim 2 further comprising a removable needle 118a adapted to be housed within the cannula 118.

4. The angiocatheter device 100 of claim 3 further comprising a needle stick safety device 41c engaged with the needle, the needle stick safety device 41c is removed from device 100 if the needle 118a is removed.

5. The angiocatheter device 100 of claim 1 further comprising a lip 128 disposed around the second end 112 of the catheter body 110.

6. The angiocatheter device 100 of claim 1 further comprising stabilizing tabs 12 disposed on and protruding from sides of the catheter body 110.

7. The angiocatheter device 100 of claim 1, wherein the catheter body 110 is generally cone-shaped with the first end 111 having a diameter smaller than that of the second end 112.

8. The angiocatheter device 100 of claim 1, wherein the catheter body 110 is generally cylindrical in shape.

9. The angiocatheter device 100 of claim 1 further comprising a window 13 disposed in the catheter body 110.

10. The angiocatheter device 100 of claim 9, wherein the window 13 is constructed from a translucent, transparent, or clear material.

11. The angiocatheter device 100 of claim 1, wherein the stationary anchor 130 is generally cone-shaped with the second end 132 having a diameter smaller than that of the first end.

12. The angiocatheter device 100 of claim 1, wherein the compressible valve 150 is constructed from a material comprising a silicone, a rubber, a foam, a copolymer, a block polymer, combination of the aforementioned materials or any other elastomeric, compressible, sterilize able, medical grade material.

13. The angiocatheter device 100 of claim 1, wherein the first end of the compressible valve 150 is mounted around the stationary anchor 130 via a mounting component 158.

14. The angiocatheter device of claim 1 further comprising a cover 59 adapted to temporarily cover the second end 15 of the hub 14.

15. The angiocatheter device of claim 14, wherein the cover 59 is attached to the catheter body 10 via an attachment means 59a.

16. The angiocatheter device of claim 1 further comprising a saline lock 59c attached to the catheter body 10 via an attachment means 59a.

17. An angiocatheter device comprising:
(a) a catheter body 10 having a first end, a second end, and a hub 14 disposed on the second end of the catheter body 10, the connection hub 14 has an open second end 15;
(b) a valve disposed in the hub 14 of the catheter body 10; and
(c) a channel 12a, the channel 12 extending from the valve to at least the first end of the catheter body 10;
wherein the valve can move between an open position and a closed position and is biased in the closed position, wherein when a needle 21a or a medical fitting 31a is introduced into the hub 14 of the catheter body 10 via the open second end 15 of the hub 14 and makes contact with the valve the valve moves to the open position allowing fluid to transfer between the channel 12a and the needle 21a or medical fitting 31a, and when the needle 21a or medical fitting 31a is removed from the valve the valve moves to the closed position preventing flow of fluid from channel 12a into the catheter body 10.

18. The angiocatheter device of claim 17, wherein the valve is a reed-type valve 26, a tines valve 36, a clapper-type valve 46, a ball-type valve, a self-sealing membrane valve, a septum valve 66, a compressible valve 150, or a combination thereof.

19. The angiocatheter device of claim 18, wherein the septum valve 66 is constructed from a material comprising a silicone, a rubber, a foam, a copolymer, a block polymer, combination of the aforementioned materials or any other elastomeric, compressible, sterilize able, medical grade material.

20. The angiocatheter device of claim 17 further comprising a cannula 11 disposed on the first end of the catheter body 10 and fluidly connected to the channel 12a.

21. The angiocatheter device of claim 20 further comprising a removable needle 21a adapted to be housed within the cannula 11.

22. The angiocatheter device of claim 21 further comprising a needle stick safety device 41c engaged with the needle 21a, the needle stick safety device 41c is removed from device if the needle 21a is removed.

23. The angiocatheter device of claim 17 further comprising stabilizing tabs 12 disposed on and protruding from sides of the catheter body 10.

24. The angiocatheter device of claim 17 further comprising a window 13 disposed in the catheter body 10.

25. The angiocatheter device of claim 17 further comprising a cover 59 adapted to temporarily cover the second end 15 of the hub 14.

26. The angiocatheter device of claim 25, wherein the cover 59 is attached to the catheter body 10 via an attachment means 59a.

27. The angiocatheter device of claim 17 further comprising a saline lock 59c attached to the catheter body 10 via an attachment means 59a.

28. The angiocatheter device of claim 18 further comprising a removable saline lock 59c attached to the catheter body 10 via an attachment means 59a.
